(19) United States
(12) Patent Application Publication
(10) Pub. No.: US 2026/0097176 A1
(43) Pub. Date: Apr. 9, 2026

(54) TUBE SUPPORT APPARATUS AND METHODS

(71) Applicant: WarriorNP LLC, Bloomfield Hills, MI (US)

(72) Inventors: Jessica Harnish, Bloomfield Hills, MI (US); Steven Fileccia, Auburn Hills, MI (US); Brian Smith, Auburn Hills, MI (US); Greg Jablonski, Auburn Hills, MI (US)

(21) Appl. No.: 19/349,402

(22) Filed: Oct. 3, 2025

Related U.S. Application Data
(60) Provisional application No. 63/703,098, filed on Oct. 3, 2024.

(51) Int. Cl. A61M 16/04

(52) U.S. Cl. CPC A61M 16/0497; A61M 2205/0216; A61M 2209/088; A61M 2210/0606

(57) ABSTRACT

A tube support apparatus is provided for stabilizing tubing relative to a patient. The apparatus includes a brace configured for attachment to the patient and a support extending from the brace. The support defines a surface having a groove for receiving a tube and a channel oriented transverse to the groove. A clamp is coupled to the support and includes a bar that secures the tube within the groove. The bar has an inner surface with a pad positioned to lie in the transverse channel when the bar is engaged.

### CROSS-REFERENCE TO RELATED APPLICATION

This application claims priority to U.S. Provisional Application No. 63/703,098, which was filed on Oct. 3, 2024.

### BACKGROUND

Endotracheal intubation is a medical procedure in which a flexible tube is placed through the mouth or nose into the windpipe (trachea) to help a patient breathe. In some instances, endotracheal intubation is an emergency procedure that is performed on a patient who may be unconscious or who cannot breathe on their own.

Nasogastric tubes or orogastric tubes are small tubes placed either through the nose or the mouth and end with the tip in the stomach or the small intestines. Nasogastric tubes or orogastric tubes may be used for feedings, medication administration, or removal of contents from the stomach via aspiration, suction, or gravity drainage.

Gastric suction is a procedure to empty the contents of your stomach. Gastric suction is performed to empty the contents of the stomach before it passes through the rest of the digestive tract.

### SUMMARY

In some aspects, the techniques described herein relate to a tube support apparatus, including a brace for attachment to a patient. A support extends from the brace and includes a surface providing a groove for receiving a tube and a channel transverse to the groove. A clamp includes a bar for securing the tube within the groove, and the bar includes a pad on an inner surface of the bar positioned to lie in the channel when the bar is in an engaged position.

In some aspects, the techniques described herein relate to a tube support apparatus, and the brace includes a curved support portion having opposed ends, each end received in a respective pad assembly adhered to the patient.

In some aspects, the techniques described herein relate to a tube support apparatus, and the support includes superior and inferior surfaces, each surface providing a groove for receiving respective tubes.

In some aspects, the techniques described herein relate to a tube support apparatus, and the bar is pivotable about a hinge axis substantially parallel to the groove.

In some aspects, the techniques described herein relate to a tube support apparatus, and the clamp includes a polymeric material.

In some aspects, the techniques described herein relate to a tube support apparatus, and the support includes a superior surface providing a plurality of grooves for receiving respective tubes and an inferior surface providing a plurality of grooves for receiving respective tubes, such that a tube on one surface can be adjusted while a tube on the opposite surface remains secured.

In some aspects, the techniques described herein relate to a tube support apparatus, and the grooves on the superior surface are sized differently from the grooves on the inferior surface to accommodate tubes of different diameters.

In some aspects, the techniques described herein relate to a tube support apparatus, and the clamp includes the bar configured to secure a tube within the groove on the superior surface and a second bar configured to secure a tube within a second groove on the inferior surface, the bar being movable independently of the second bar.

In some aspects, the techniques described herein relate to a tube support apparatus, and movement of the bar allows selective release or adjustment of a tube in a groove of the superior surface while a tube in the second groove of the inferior surface remains secured.

In some aspects, the techniques described herein relate to a tube support apparatus, and the brace includes a curved portion contoured to complement the patient's face.

In some aspects, the techniques described herein relate to a tube support apparatus, and the clamp includes the bar configured to secure a tube within a groove on the superior surface and a second bar configured to secure a tube within a groove on the inferior surface, the bars being pivotable about hinge axes substantially parallel to the grooves and independently movable to selectively release or adjust a tube.

In some aspects, the techniques described herein relate to a tube support apparatus, further including a second groove on the support and a second channel transverse to the second groove, and the clamp further includes a second bar for securing a tube within the second groove, the second bar including a second pad configured to lie in the second channel when the second bar is in an engaged position.

In some aspects, the techniques described herein relate to a tube support apparatus, and the bar and the second bar are pivotable about hinge axes substantially parallel to the grooves and independently movable to selectively release or adjust a tube.

In some aspects, the techniques described herein relate to a tube support apparatus, and the brace includes a curved support portion with opposed ends received in cheek pad assemblies adhered to the patient, the support further including a second groove on an opposed surface and a second channel transverse to the second groove, and the clamp further including a second bar for securing a tube within the second groove, the second bar including a second pad on an inner surface of the second bar configured to lie in the second channel when the second bar is in an engaged position, the bar and the second bar being pivotable about hinge axes substantially parallel to the first and second grooves and independently movable to selectively release a tube secured by one of the first bar and the second bar while another tube secured by the other of the first bar and the second bar remains secured.

In some aspects, the techniques described herein relate to a tube support apparatus, and the pad includes an elongated precut self-adhesive piece of medical foam rubber.

In some aspects, the techniques described herein relate to a tube support apparatus, and the pad includes a compressible material selected from foam, gel, silicone, rubber, or elastomer, configured to conform to an outer surface of the tube.

In some aspects, the techniques described herein relate to a tube support apparatus, and a first tube is secured in a groove on the superior surface and a second tube is secured in a groove on the inferior surface, the clamp being configured such that the first tube can be adjusted longitudinally while the second tube remains fixed.

In some aspects, the techniques described herein relate to a tube support apparatus, and the support and the clamp are molded as a single piece with the bar formed as a living hinge.

In some aspects, the techniques described herein relate to a tube support apparatus, and the bar is secured in the engaged position by a bolt and eye arrangement, the bolt being provided on one of the support and the bar and the eye being provided on the other of the support and the bar.

In some aspects, the techniques described herein relate to a tube support apparatus, and the bar is secured in the engaged position by a bolt extending from the support and received in an eye formed in the bar.

These and other features may be best understood from the following specification and drawings, the following of which is a brief description.

### BRIEF DESCRIPTION OF THE DRAWINGS

FIG. 1 is a perspective view of an example tube support apparatus.

FIG. 2 is a perspective view of another example tube support apparatus.

FIG. 3 is an enlarged view showing an example end portion received in a channel of an example cheek pad assembly.

FIG. 4 is a perspective view of an example cheek pad.

FIG. 5 illustrates an example end portion.

FIG. 6 is a schematic cross-sectional view of an example cheek pad channel.

FIG. 7 is a perspective view of another example support apparatus.

FIG. 8 illustrates an example cheek pad and end portion of the example tube support apparatus of FIG. 7.

FIG. 9 illustrates another view of the example cheek pad of FIG. 8.

FIG. 10 illustrates the example tube support apparatus of FIG. 7.

FIG. 11 illustrates another view of the example cheek pad and end portion of the example tube support apparatus of FIG. 7.

FIG. 12 is a perspective view of another example support apparatus.

FIG. 13 is a front view of the support apparatus of FIG. 12.

FIG. 14 is another view of the support apparatus of FIGS. 12 and 13, with the example curved portion removed for ease of viewing.

FIG. 15 is another view of the support apparatus of FIGS. 12-14, with the example curved portion removed for ease of viewing.

FIG. 16 is another view of the support apparatus of FIGS. 12-15, with the example curved portion removed for ease of viewing.

FIG. 17A is another view of the support apparatus of FIGS. 12-16 in an engaged position.

FIG. 17B is another view of the support apparatus of FIGS. 12-16 in an engaged position with tubes.

FIG. 17C is a front view of the support apparatus of FIGS. 12-16, with the example curved portion removed for simplified viewing.

FIG. 18 is another view of the support apparatus of FIGS. 12-17B with a bar in a disengaged position.

FIG. 19 is a perspective view of another example support apparatus.

FIG. 20 is another perspective view of the example support apparatus of FIG. 19.

FIG. 21 is another view of the example support apparatus of FIGS. 19-20.

FIG. 22 is another view of the example support apparatus of FIGS. 19-21.

FIG. 23A is a front view of another example support apparatus.

FIG. 23B is a front view of the example support apparatus in an engaged position, with the example curved portion removed for ease of viewing.

FIG. 24A is a top view of another example support apparatus.

FIG. 24B is a perspective view of the example support apparatus of FIG. 24A.

### DETAILED DESCRIPTION

This application is related to apparatuses and methods for supporting endotracheal, feeding, gastric suction, and/or other tubes. In some prior art apparatuses, tape is used to secure the tube, such as an endotracheal tube, to a brace. Applicant has identified certain disadvantages of using tape. When infants are intubated and premature, they are often subjected to humidity for 30 or more days, which can make the tape less effective at securing and possibly lead to the tube coming out of position. Another drawback of tape is that medical professionals often have to remove their gloves before taping, removing the tape, or adjusting the tape, adding time and difficulty to the procedure. Still another drawback of tape is that intubated infants often vomit, which also can make the tape less effective at securing if the vomit contacts the tape. Yet another drawback of these prior art devices is that they often require 2-3 medical professionals to perform adjustments.

The example clamps and associated apparatuses and methods disclosed herein overcome the Applicant-identified disadvantages with prior art apparatuses, including those that use tape. For example, clamps with polymeric materials, including elastomers, for securing a tube to a brace are relatively unaffected by humidity and vomit. Further, clamps with polymeric materials are easy to quickly adjust. In some examples, the clamps may lack metal so that patients can undergo procedures such as magnetic resonance imaging while remaining intubated. In some examples, the braces, clamps, and/or supports disclosed herein may be plastic. In some examples, the braces, clamps, and/or supports disclosed herein may be made of medical grade polypropylene. Further, the examples disclosed herein allow a single medical professional to make an adjustment. Examples disclosed further eliminate the need for paper or plastic tape measures to be placed against the lip of the patient for measuring and adjusting tube depth.

Further, the examples disclosed herein allow multiple tubes to be secured with one clamp at once, and further allow one of those tubes to be selectively removed or adjusted while the other tube remains secured, i.e., held in place longitudinally to control the depth of the tube in the patient. The benefits described herein are not limited to intubation procedures and may be achieved by other procedures, including feeding or gastric suction tube procedures. Further, while procedures regarding infants are described in some of the example embodiments, procedures for other patients of various ages may benefit from this disclosure.

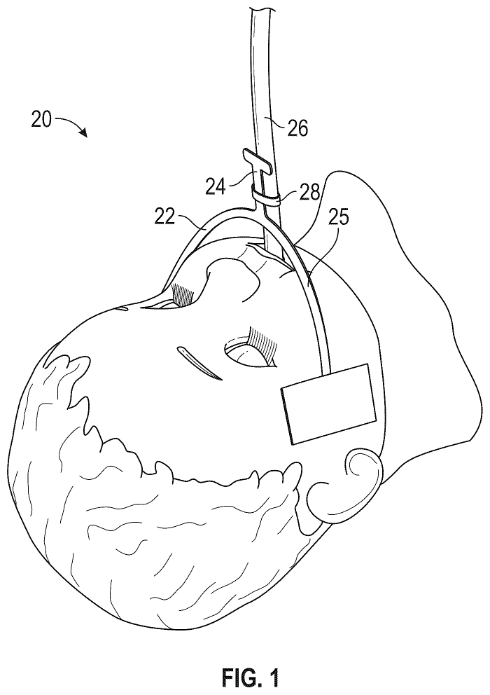

FIG. 1 illustrates an example tube support apparatus 20 including a brace 22 for attachment to a patient's face. The brace 22 may include a support 24 extending away from the patient's face as shown. The example support 24 extends from a curved portion 25 that is contoured to complement the patient's face and secured to the patient's face at its ends. A tube 26, such as an endotracheal tube, is received against the support 24 and placed through the mouth of the patient. A clamp 28 is provided to secure the tube 26 to the support 24 and surrounds both the tube 26 and the support 24. The example clamp 28 is made of one or more polymeric materials. The term polymer is used herein to refer generally to plastics, elastomers, thermoplastic elastomers, or other natural or synthetic polymeric materials, including silicones and polyurethanes, that contain repeating molecule subunits. In some examples, the clamp 28 is plastic.

In the example shown, the patient is a newborn child, but intubations of other patients of various ages may benefit from this disclosure. Because of the small size of newborn children, the slightest adjustment in position of the tube can mean the difference of the infant receiving oxygen and not receiving oxygen. Accordingly, the examples disclosed herein provide improved securement of endotracheal tubes. Additionally or alternatively, procedures involving nasogastric tubes, orogastric tubes, and gastric suction may benefit from this disclosure, where small shifts can affect the delivery of nutrition, medication, or suction.

In some examples, the tube support apparatuses disclosed herein, including the example clamps, are made of medical grade polypropylene.

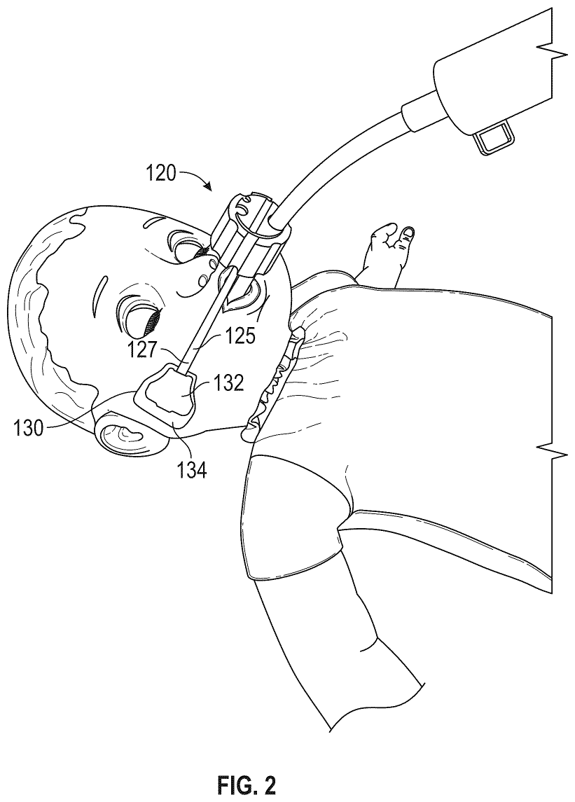

FIG. 2 illustrates another example tube support apparatus 120 in which the end portions 127 of the curved portion 125 are received in a pad assembly 130, and more specifically in a receiver pad 132 adhered to an adhesive pad 134 adhered to the patient's skin proximal to the ear. It should be understood that like reference numerals identify corresponding or similar elements throughout the several drawings. In some examples, such as in infants, this area offers the most surface area for adhesion. In some examples, the pad assembly 130 is adhered in a position closer to the patient's ear than to the patient's mouth.

Although one end of the curved portion is shown in one pad assembly 130 in the Figure, a similar arrangement is utilized proximal to the patient's other ear. In some examples, as shown, both the receiver pad 132 and the adhesive pad 134 are tapered, narrowing as they extend in the direction from the patient's ear to the mouth (medially), such as like the tapered pads previously discussed in this disclosure. In some examples, the receiver pad 132 includes silicone. In some examples, the adhesive pad 134 is a silicone tape adhesive pad. In some examples, the adhesive pad 134 is an acrylic adhesive pad. The example adhesive pad 134 may be larger than the example receiver pad 132 in some examples.

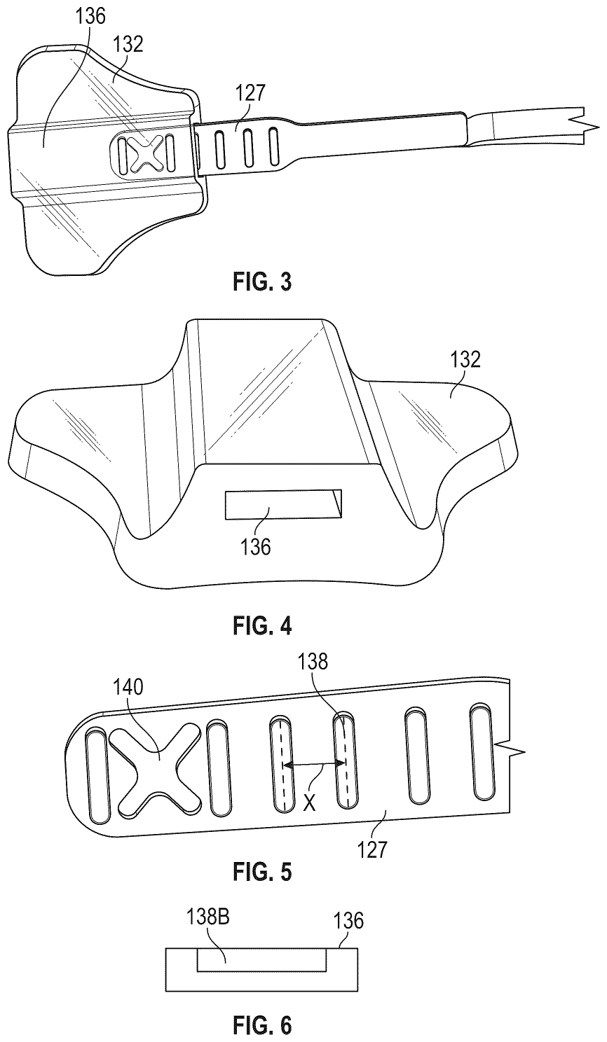

As shown in FIGS. 3 and 4, the end portion 127 is received in a channel 136 of the receiver pad 132. The end portion 127 is adjustable within the channel 136 to allow the example support apparatus 120 to be placed in an optimal position.

As shown in FIG. 5, the end portion 127 may include protrusions 138, such as ribs in some examples, for securement of the end portion 127 within the channel 136 (FIGS. 3 and 4). In some examples, as shown schematically in FIG. 6, the channel 136 may include one or more similar protrusions 138B, such as on its upper surface in some examples, for engaging with protrusions 138 on the end portion 127 and securing the end portion 127 in position once an optimal position is attained. An indicator 140 may be included on the end portion 127 to signal to a medical professional when to stop pulling the end portion 127 out of the channel 136 during an adjustment process to prevent the medical professional from pulling the end portion 127 completely out of the channel 136.

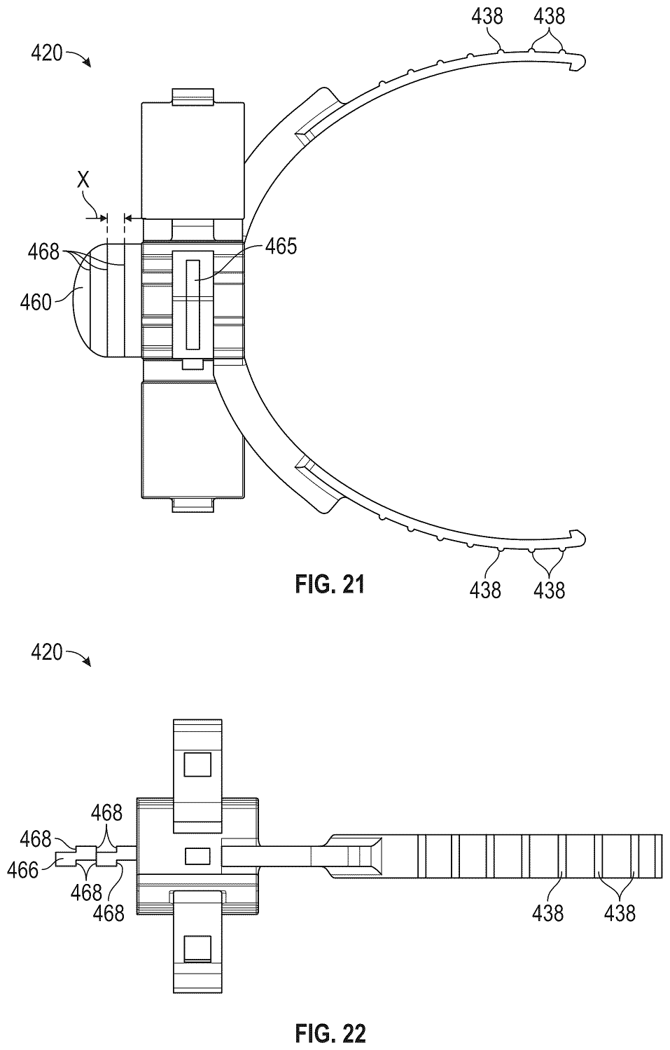

In some examples, the protrusions or ribs 138 along the end portion 127 are spaced at uniform increments, such as 0.25 cm in some examples, though other values may be utilized, thereby serving as indicia for controlled repositioning of the arc within the cheek pad channels 136. The ribs 138 may cooperate with corresponding protrusions or detents 138B in the cheek pad channel 136 to provide tactile and visual feedback during adjustment. With reference to FIGS. 21 and 22, which will be discussed in further detail, in some implementations, the indicators 468 on the tab 466 are spaced at the same pitch as the ribs 438, enabling a clinician to adjust the tube and the curved portion 425 by the same number of increments.

Regarding the example support apparatus 120 or any other apparatus that uses the pads 130, in an emergency, the brace can simply be pulled up and out of the pads 130. The pads 130 can remain on the patient's face until the emergency has been resolved. The brace can then be replaced into the pads 130 and reused. This is an improvement over some prior art devices which suggest the device be cut with scissors and pulled off the patient's face. Further, the example pads 130 are able to be cleaned with baby wipes or alcohol pads. Prior art pads could become soiled with vomit and/or saliva because they are cotton-topped, and the whole apparatus would need to be changed out.

With reference to FIGS. 2-6, an example method may include one or more of the following steps: intubating a patient, placing a pad assembly 130 on the patient's cheek proximal to the patient's ear, placing a second pad assembly 130 on the patient's cheek proximal to the patient's other ear, positioning the end portions 127 of the curved portion 125 within the channels 136 of each pad assembly 130, adjusting the end portions 127 within the channels 136 of each pad assembly 130 to an optimal brace position, and securing the endotracheal tube within the example support apparatus 120. Although an example support apparatus 120 is shown, other apparatuses, including other examples in this disclosure, may be utilized with the pad assembly 130. In some examples, there is no need for an additional adhesive to be placed over the top of the receiver pad 132. Fewer or additional steps than are recited could be performed within the scope of this disclosure, and the recited order of steps is not intended to limit this disclosure.

In some examples, the grooves receiving the tubes may have indicators, such as notches or raised portions in some examples, for reference against the tubes for adjustment and positioning of the tubes. In some examples, the indicators may be spaced apart in equal intervals. In some examples, the intervals may be 0.25 cm.

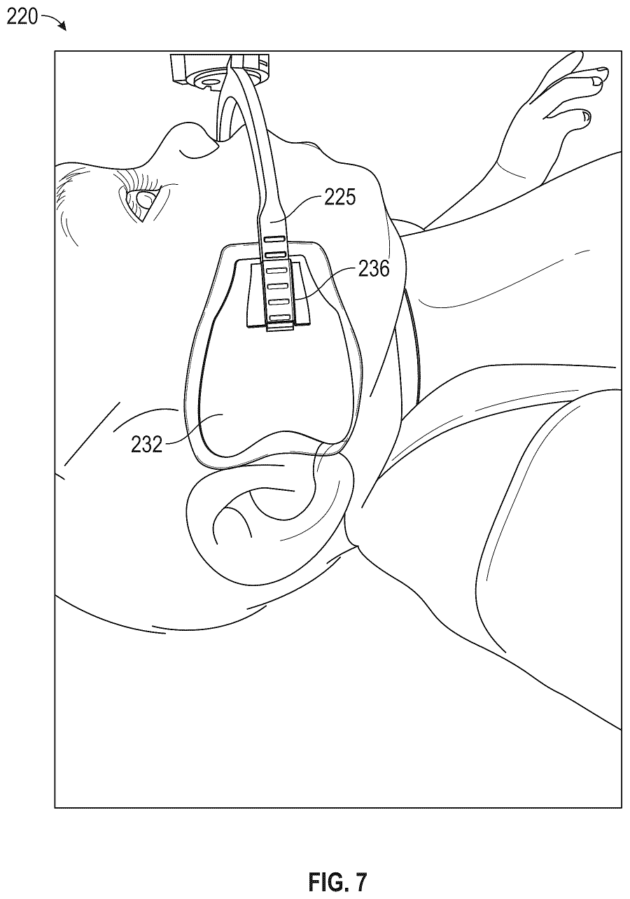

FIG. 7 illustrates another example support apparatus 220. In some examples, as shown, the channel 236 may not extend to the lateral end of the cheek pad 232. At least a portion of the end portion 225 may extend laterally beyond the channel 236 while secured.

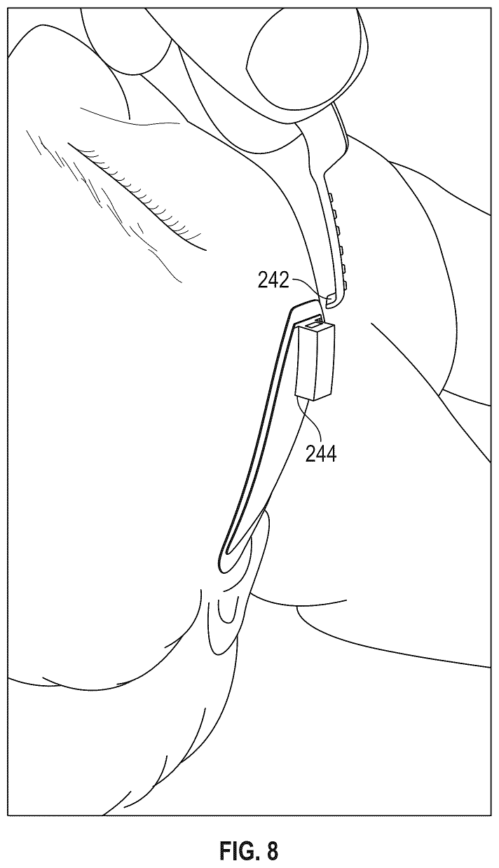

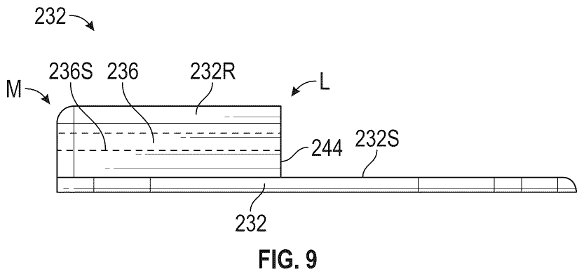

As shown in FIGS. 8 and 9, a raised edge 244 relative to the surrounding surface of the cheek pad 232 is therefore provided at the lateral end of the channel 236.

As shown in FIG. 9, the channel 236 may be a through opening extending from the medial end M to the lateral end L of the raised portion 232R of the cheek pad 232. The raised portion 232R is raised relative to a main body portion 232M. The lower surface 236S is raised relative to the upper surface 232S of the main body portion 232M of the cheek pad 232, providing the raised edge 244 at the lateral end L. The edge 244 may extend from the lateral end of the channel 236 to the surface 232S of the main body portion 232M. Other edges, including edges at the lateral end of the raised portion 232R may be used to engage a support apparatus in other examples. The main body portion 232 may extend laterally farther than the raised portion 232R and channel 236, as shown.

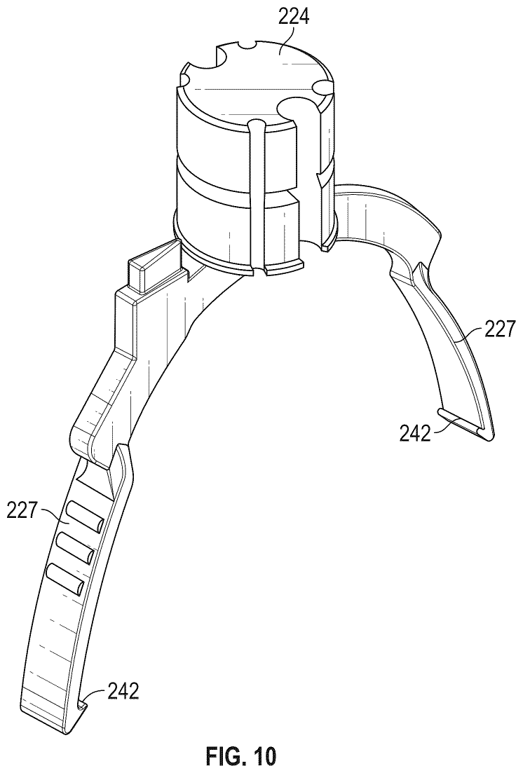

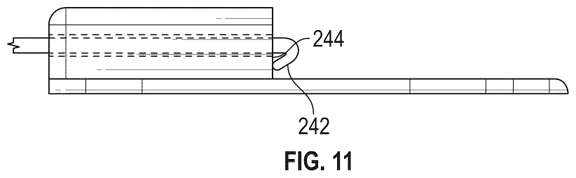

As shown in FIGS. 10 and 11, a protrusion 242 extends from one or both lateral ends of the end portion 227, so as to engage the raised edge 244 and inhibit the end portions 227 from being pulled out of their respective channels 236 under a certain threshold force. In some examples, as shown, the protrusion 242 may be angled to extend back toward the support 224 so as to hook the raised edge 244. In some examples, the protrusion 242 and raised edge 244 are configured such that the infant patient cannot pull the end portion 227 out of the channel 236 but a medical professional can if desired. In some examples, a protrusion may be configured to engage a different edge at the lateral end L of the channel than the one shown in the illustrative example.

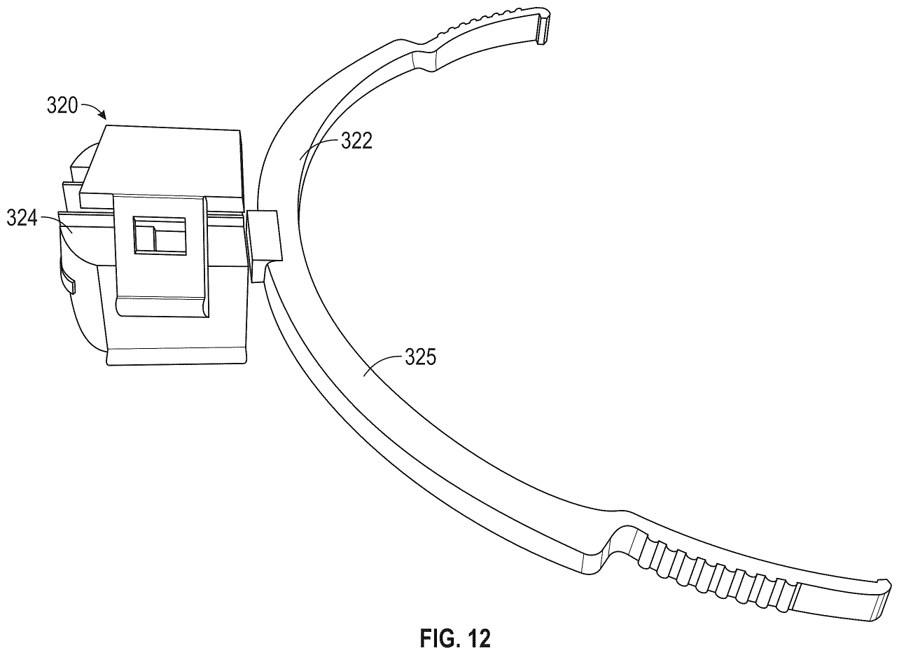

FIGS. 12-18 illustrate another example support apparatus 320 including a brace portion 322 for attachment to a patient's face. It is contemplated that other similar apparatuses could attach to other parts of a patient. A support 324 extends from the brace portion 322 and away from the patient's face as shown. The example support 324 extends from a curved portion 325 that is contoured to complement the patient's face and secured to the patient's face at its ends, such as by the example pads disclosed herein (e.g., FIGS. 3-9) in some examples. One or more tubes (not shown) may be received against the support 324 and may be inserted into the patient, such as through the mouth of the patient in an intubation process in some implementations.

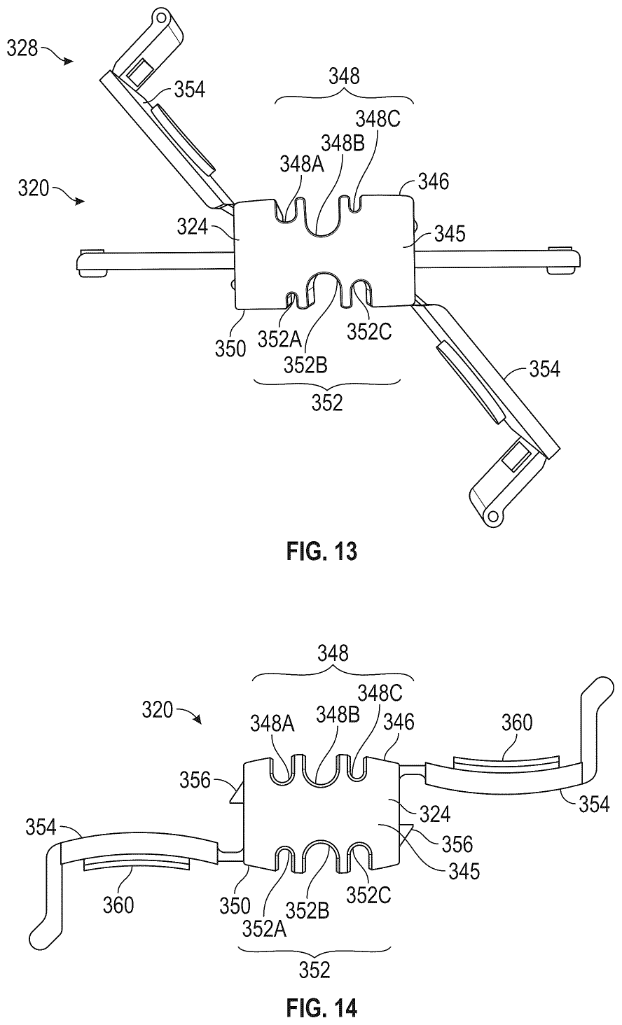

One or more surfaces of the support 324 may be contoured to receive one or more tubes. In some implementations, two opposed surfaces of the support 324 may be configured to receive respective tubes. For example, as shown in FIGS. 13 and 14, the support 324 includes a superior facing surface 346 and/or an inferior facing surface 350 for receiving one or more tubes. In some implementations, as shown, the superior facing surface 346 provides one or more grooves 348A, 348B, 348C for receiving one or more tubes (not shown), and the inferior facing surface 350 provides one or more grooves 352A, 352B, 352C for receiving one or more tubes (not shown).

In some examples, the grooves 348, 352 are sized to accommodate tubes of different diameters, including an endotracheal tube, an orogastric tube, and/or a nasogastric tube in some examples. As used herein, the term “groove” is intended to be interpreted broadly to encompass any surface feature configured to receive or retain a tube, including but not limited to channels, slots, recesses, indentations, or equivalent structures, regardless of orientation, shape, or cross-section. In some implementations, one or more of the grooves 348, 352 may be substantially U-shaped, although other shapes such as V-shaped, arcuate, or irregular cross-sections may also be used. In further implementations, the grooves 348, 352 may be straight, curved, angled, or segmented, and may be formed integrally with the support or provided by a separate insert, liner, or coating.

A clamp 328 is provided to secure the one or more tubes to the support 324. In some implementations, the clamp 328 includes one or more clamp elements, such as one or more bars 354, that are independently movable between engaged and disengaged positions relative to the support. In the engaged position, the respective bar 354 bears against, overlaps, or otherwise engages one or more tubes received within the grooves 348, 352 to secure the one or more tubes in place. The clamp elements may move by pivoting, sliding, snapping, deforming, or other relative movement to transition between the engaged and disengaged positions.

The one or more bars 354 may be pivotable between engaged and disengaged positions. One or both of the bars 354 may be pivotable about one or more hinges or joints such that the bar 354 swings toward the support to capture a tube and away from the support to release the tube. The one or more bars 354 may be movable between engaged and disengaged positions in other ways in some implementations. For example, the one or more bars 354 may be detachable from the support 324. Additionally or alternatively, sliding or snapping mechanisms could be used to secure the bars 354.

In some implementations, the support 324 includes a main body block portion 345 that defines one or more surfaces for supporting tubes. The block portion 345 provides the surfaces 346, 350 on which grooves 348, 352 may be formed. The clamp bars 354 are configured to hinge, pivot, or otherwise move into and away from contact with the block portion 345 so as to selectively secure or release tubes received in the grooves 348, 352. The block portion 345 may be of any suitable geometry that permits the formation of tube-receiving regions and engagement by the clamp bars 354, including but not limited to regular or irregular, curved or planar, or composite forms.

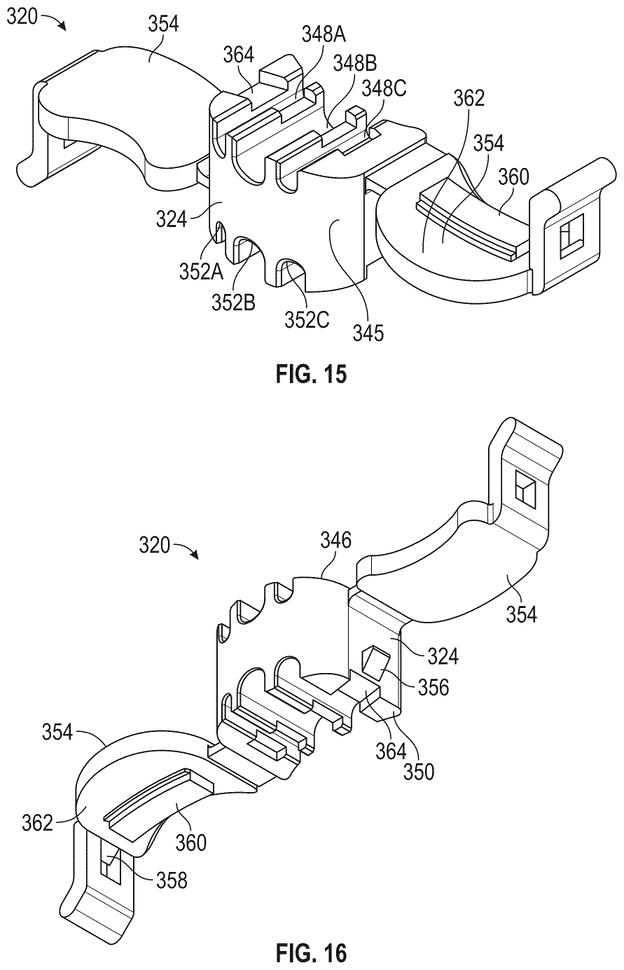

In some implementations, as shown in FIG. 16, one or more of the bars 354 may latch to the support 324 to secure the bar 354 in the engaged position, such as by a snap-tab closure. As one example, a bolt 356 may extend from the support 324, which may be received in an eye 358 in the bar. The example bars 354 may be substantially L-shaped, though one or both legs of the L may have a curvature in some examples. The example bars 354 may hinge from opposite lateral sides of the support 324. The hinge axes may be substantially parallel to the grooves 348A, 348B, 348C, 352A, 352B, 352C (see FIG. 15). As used herein, the term “substantially parallel” refers to an orientation that is parallel within typical manufacturing tolerances, such as within ±10 degrees of true parallel.

The example bars 354 may be arranged in a reverse swing configuration as shown, such that the bars 354 swing outward from the support 324. Other configurations are contemplated. In some examples, the bolt may be provided on the support 324 and the eye on the bar 354, or vice versa, with the bolt and eye arrangement configured to secure the bar in the engaged position.

A pad 360 may be provided at the inner surface 362 of one or more of the bars 354. The pad 360 may comprise a cushioning element formed of a compliant, biocompatible material, such as foam, gel, or elastomer, sized to contact a tube when the bar is engaged. The pad 360 may take the form of a strip or insert of resilient material disposed along the inner surface 362 of the bar to distribute pressure and increase friction against a tube. The pad 360 may be provided as a compressible interface, for example a piece of medical-grade foam, silicone, or rubber, configured to conform to the outer surface of a tube. In implementations, as shown, the pad 360 may protrude or be raised relative to the adjacent inner surface 362 of the bar, such that the pad 360 projects inwardly toward the tube to increase contact pressure and retention.

In some examples, the pad 360 may be an elongated precut self-adhesive piece of medical foam rubber. A corresponding channel 364 may lie in one or both of the surfaces 346, 350 transverse to the grooves 348A, 348B, 348C, 352A, 352B, 352C and for receiving the pad 360 when the bar 354 is in the engaged position.

The channel 364 may be recessed below the surrounding surface but raised relative to the grooves so that the pad 360 projects toward the tubes while still being seated within the surface. The channel 364 may have less depth than its corresponding grooves 348A, 348B, 348C, 352A, 352B, 352C. In other examples, the channel 364 may be equal in depth or deeper, depending on the desired retention and cushioning effect. In some examples, when the bar 354 is L-shaped, the pad 360 may be disposed along an inner surface of one leg of the L-shape, and the eye 358 may be formed in the other leg of the L-shape.

When the one or more bars 354 are secured in the engaged position, the pad 360 (e.g., foam rubber, silicone, or another compliant material) may compress in contact with the one or more tubes. Such compression may increase static friction and conform to the outer surface of the tube to enhance securement. The channel 364 may receive a portion of the pad 360 not in contact with the tube, so that the pressure applied by the bar 354 through the pad 360 is directed primarily onto the tube rather than distributed across the adjacent surface 346, 350. In some implementations, the channel 364 may also help maintain pad alignment and concentrate compressive force on the tube to further resist displacement.

In some implementations, the support 324 and clamp 328 may be molded as a single piece, with the bars 354 formed as living hinges and secured by a snap-tab closure. Other variations are contemplated, including configurations in which the bars 354 are detachable from the support 324 or otherwise re-attachable. The bars 354 may be repeatedly and independently opened and closed as needed to secure, release, or adjust one or more tubes. In some examples, the bars 354 may be formed of a polymeric material, such as plastic, elastomer, or composite. One or both of the support 324 and clamp 328 may likewise be formed of a polymeric material, although metallic or hybrid constructions are also contemplated. In further implementations, the support and clamp may be manufactured by molding, additive manufacturing, or machining, and may incorporate combinations of rigid and flexible regions to achieve the desired balance of strength, durability, and adjustability.

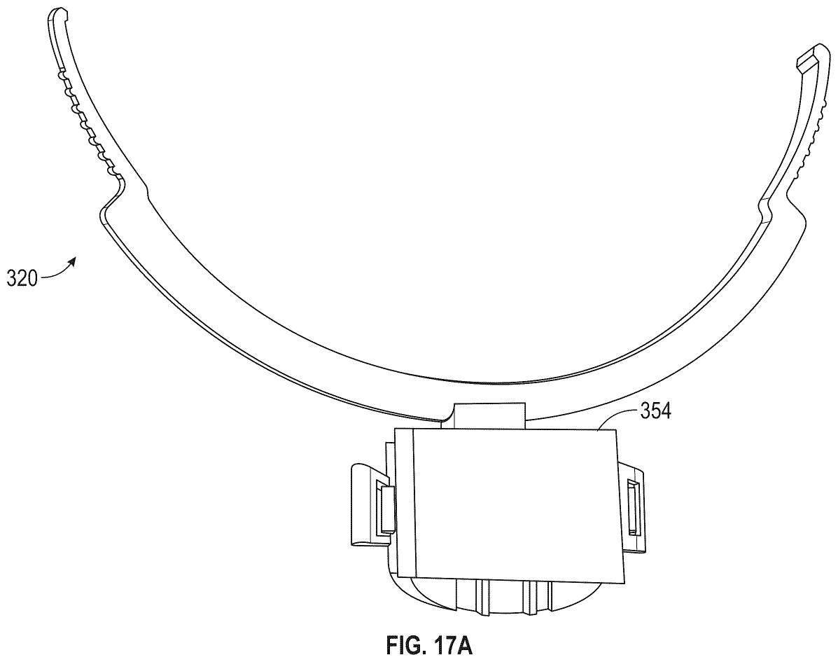

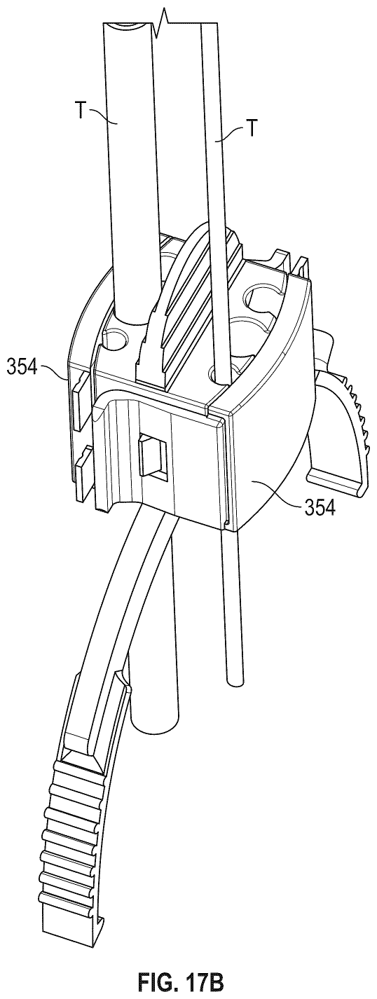

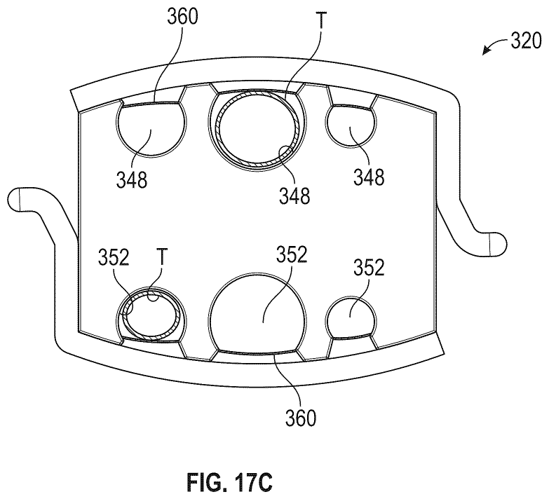

FIG. 17A shows an example bar 354 in an engaged position for securing one or more tubes (not shown) in place. FIG. 17B shows two example bars 354 in an engaged position for securing tubes T in place. Although one tube is depicted secured by each bar 354 for clarity, in some implementations, each bar 354 may be configured to secure multiple tubes simultaneously, such as two or more tubes received within adjacent grooves.

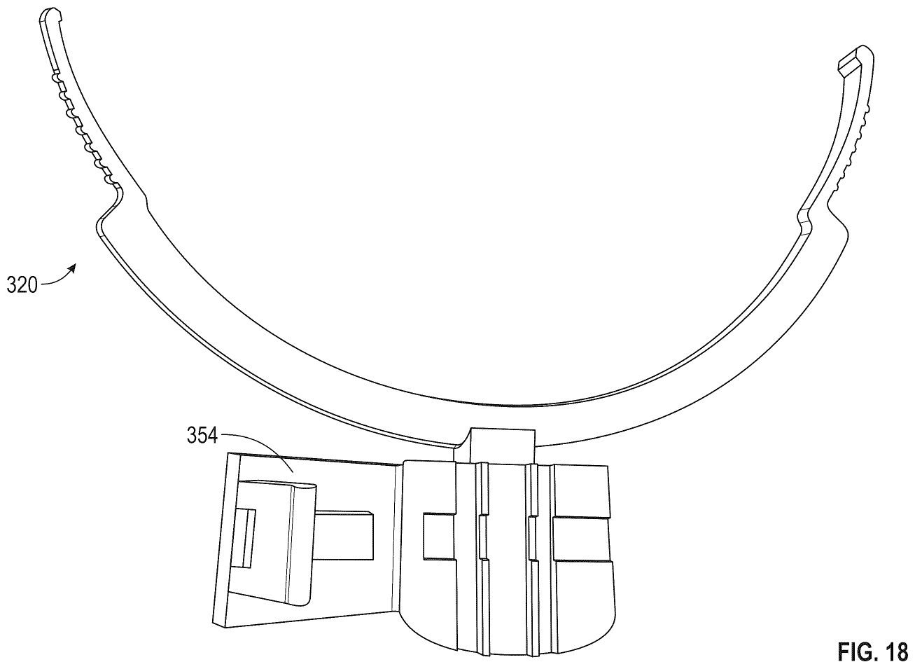

FIG. 18 shows an example bar 354 in a disengaged position. In embodiments including two or more bars 354, each bar may be selectively movable between the engaged and disengaged positions such that tubes secured by one bar 354 may be released or adjusted while tubes secured by another bar 354 remain retained. This arrangement permits independent adjustment of certain tubes without disturbing others that are already secured.

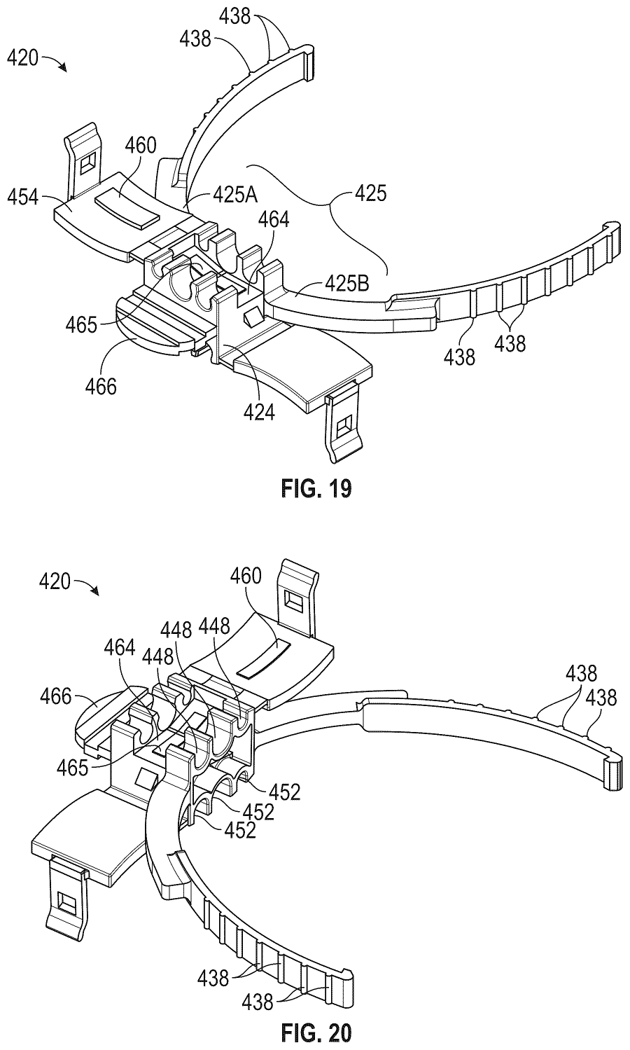

FIGS. 19-22 illustrate another example support apparatus 420 similar to the support apparatus 320. A tab 466 extends proximally from the support 424, and the curved portion 425 may include two segments 425A, 425B extending distally and laterally from the support 424. Visual cues may be utilized to facilitate tube adjustment. In some examples, the tab 466 includes one or more indicators 468 (see FIGS. 21 and 22) extending in a direction transverse to the grooves 448, 452 and/or the tubes they receive (not shown). In some implementations, the indicators 468 may be transversely extending edges spaced apart a known distance to indicate incremental adjustment distances of the tubes being held within the grooves 448, 452. In some implementations, the distance may be 0.25 cm to indicate 0.25 cm incremental adjustments of the one or more tubes. The indicators 468 may extend substantially perpendicular to the grooves 448, 452. The tab 466 may also serve as a rest to prevent kinking of the tubes in some implementations.

In some examples, as shown in FIG. 22, each of the superior and inferior surfaces of the tab 466 are staggered so as to provide indicators 468 on each surface to enhance visibility and facilitate tube adjustment.

The indicators 468 on the tab 466 may be matched in spacing to the ribs 438 on the end portion, for example both being set at 0.25 cm intervals. This correspondence permits the clinician to adjust the tube by N increments on the tab 466 and reposition the curved portion 425 by the same N ribs within the cheek pad channels (see, e.g., FIGS. 3-6), thereby relieving or preventing cheek pressure while maintaining the intended tube depth relationship.

The repositioning of the end portions within the cheek pad channels can correlate to the repositioning of the one or more tubes being supported. In some implementations, if each end portion of the curved portion 425 is advanced into its respective cheek pad channel by one rib 438 (corresponding to a distance X), the tube may be retracted by a corresponding distance X to maintain the intended insertion depth. In some examples, X can be 0.25 cm.

In some implementations, the repositioning of the end portions may involve retraction within the cheek pad channels. For example, if each end portion of the curved portion is retracted by one or more ribs (corresponding to a distance X), the tube may be advanced by a corresponding distance X.

In some scenarios, the tube is adjusted before repositioning the end portions. Although indicators 468 are shown in this illustrative example, other indicia for monitoring tube adjustment may be utilized.

The disclosed systems and methods may provide clinical benefits in scenarios in which the patient's facial contours change over time, such as in cases of sepsis, kidney failure, blood pressure instability, or dependent edema. In such circumstances, the disclosed systems and methods can allow the curved portion to be repositioned within the cheek pad channels to relieve or prevent cheek pressure while maintaining device stability, without removal or repositioning of the cheek pads. This adjustability enables clinicians to keep the curved portion as close to the cheeks as possible without applying pressure, improving comfort and safety.

Referring back to FIG. 19, in some implementations, as shown, the channel 464 may be deeper than the channel 364 (see FIG. 15) and may include a pad 465 adhered or otherwise received within the channel 464. The pad 465 may be received against an opposite surface of the tubes from the pad 460 when the bar 454 is in the engaged position. In such implementations, pads are received against the tubes on opposite surfaces of the tubes, such that a tube received in one of the grooves 448, 452 is flanked by two pads 460, 465. In some implementations, the example channel 464 may be deeper than one or more of the associated grooves 448, 452 (see FIG. 20) to accommodate the pad 465.

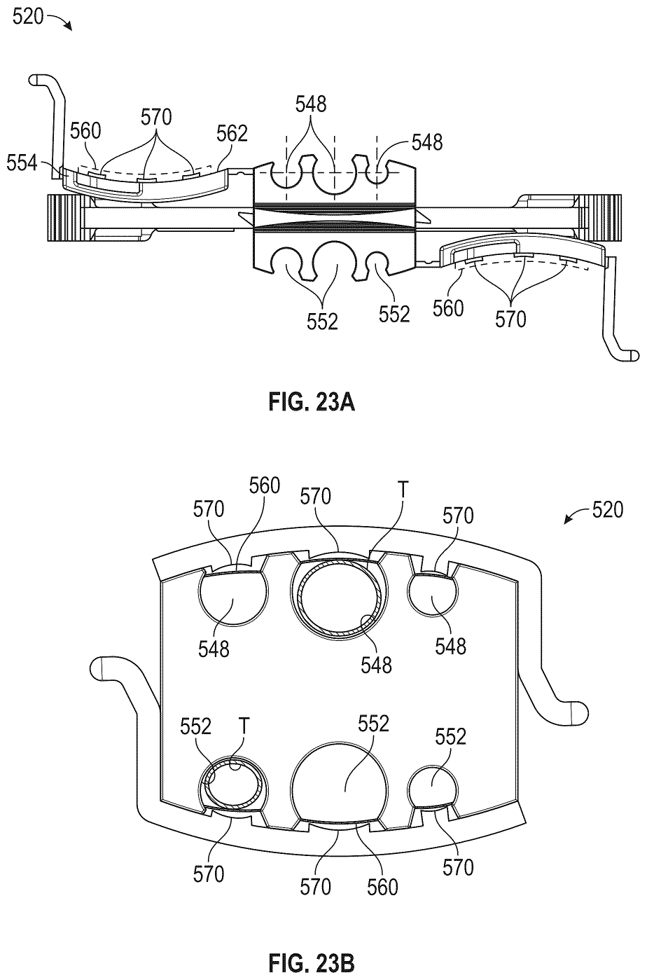

FIGS. 23A and 23B illustrate another example support apparatus 520 substantially similar to the foregoing embodiments, except that one or more projections 570 extend from an inner surface 562 of the bar 554. In some examples, the projections 570 may take the form of pegs, posts, protrusions, ribs, or the like, and are positioned beneath the pad 560 (shown dashed in FIG. 23A for illustrative purposes).

The projections 570 may be arranged to align with corresponding grooves 548, 552 of the support 524 so that, when the bar 554 is in the engaged position, the projections 570 press the pad 560 further into the grooves 548, 552. This arrangement provides additional localized compression of tubes received in the grooves 548, 552, thereby enhancing retention and reducing the likelihood of unintended tube movement in some applications.

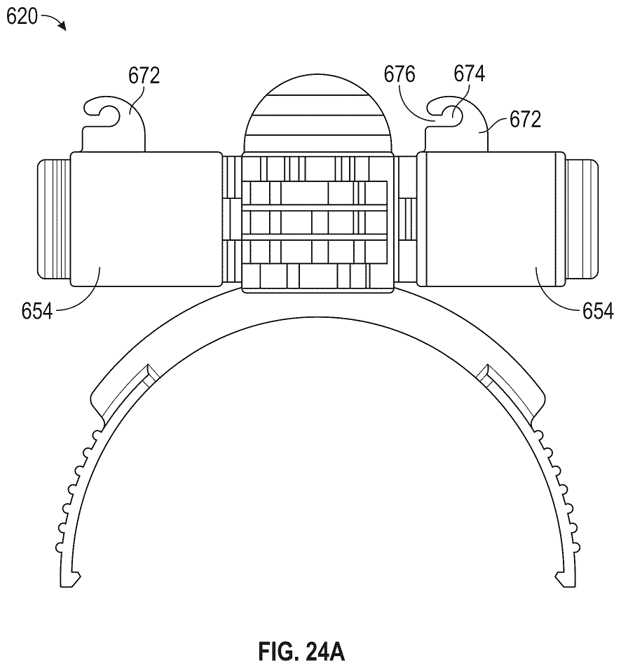

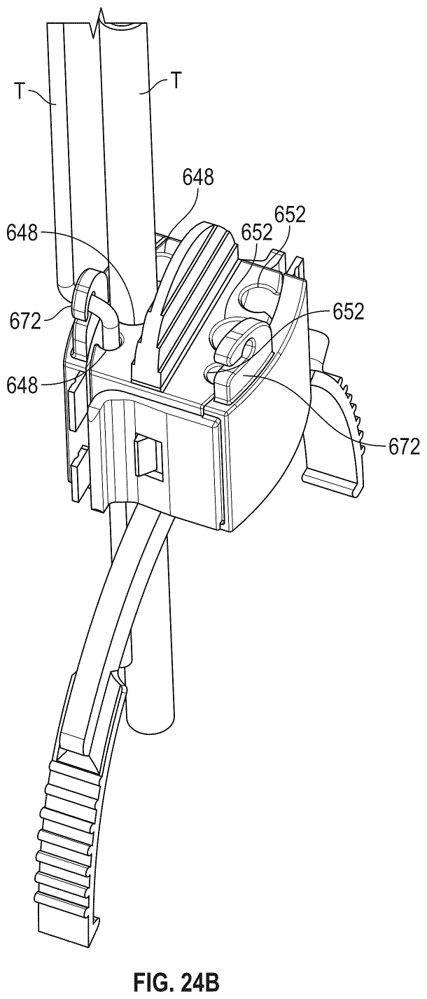

FIGS. 24A and 24B illustrate another example support apparatus 620 substantially similar to the foregoing embodiments, except that one or more hooks 672 extend proximally from one or more of the bars 654. In some examples, the hooks 672 may be integrally formed with the bars 654 and project in a proximal direction relative to the support 624.

The hooks 672 are configured to receive and secure a tube, for example by engaging a portion of the tube seated in one of the grooves 648, 652 of the support 624. In some implementations, the hooks 672 provide an additional retention feature that supplements the compression of the pad against the tube, thereby further stabilizing the tube and reducing the likelihood of displacement during use.

In some examples, the hook 672 may define an open geometry including a retention portion 674 and an entry portion 676. The retention portion 674 is configured to receive and cradle a tube and may have an enlarged diameter or cross-sectional area relative to the entry portion. The entry portion 676 is open and narrower than the retention portion so that a tube can be inserted through the entry portion 676 and into the retention portion 674, and likewise withdrawn if desired. This arrangement allows the hook 672 to securely retain the tube in the retention portion 674 while still permitting guided placement and removal of the tube through the entry portion 676. FIG. 24B illustrates an example tube received in the hook 672.

In some aspects, the techniques described herein relate to a tube support apparatus, including: a brace for attachment to a patient's face; a support extending from the brace and including a surface providing a groove for receiving a tube; a clamp including a polymeric material, including a bar for securing the tube within the groove, and the bar includes a pad on an inner surface of the bar configured to lie in a channel transverse to the groove when the bar is in an engaged position.

In some aspects, the techniques described herein relate to an endotracheal tube support apparatus, including: a brace for attachment to a patient's face; a support extending from the brace and including a superior surface providing a first groove for receiving a tube, and an inferior facing surface providing a second groove configured to receive a second tube; a clamp including a polymeric material, including a first bar pivotable about a first pivot joint for securing the tube within the first groove, and a second bar pivotable about a second pivot joint for securing the second tube within the second groove. In some implementations, the first pivot joint and the second pivot joint are on opposite lateral sides from one another.

In some aspects, the techniques described herein relate to a tube support apparatus, including: a brace for attachment to a patient's face; a support extending from the brace and including a superior surface providing a first groove for receiving a tube, and an inferior facing surface providing a second groove configured to receive a second tube; a clamp including a polymeric material, including a first bar movable between an engaged position for securing the tube within the first groove and a disengaged position, and a second bar movable between an engaged position for securing the second tube within the second groove and a disengaged position.

In some aspects, the techniques described herein relate to a tube support apparatus, including: a mounting portion configured to be secured to a patient; a projecting portion extending from the mounting portion and configured to receive one or more tubes; one or more tube-receiving regions defined on one or more surfaces of the projecting portion; one or more movable clamp arms hingedly or otherwise operably connected to the projecting portion; one or more cushioning inserts or contact surfaces disposed along inner faces of the clamp arms; and a cross-channel oriented transverse to the tube-receiving regions and positioned to align with the cushioning insert in use.

An example method of intubating a patient may be said to include attaching a brace to the patient's face. The brace may include a support extending away from the patient's face. The example method may include clamping an endotracheal tube to the support with a clamp that surrounds the endotracheal tube and the support, and the clamp may include a polymeric material.

An example method of securing a tube to a patient may include attaching a brace to the patient, such as by adhering opposed pad assemblies to the patient's face and receiving opposed ends of a curved support portion of the brace in the pad assemblies. The method may include positioning a tube against a support extending from the brace, the support including one or more grooves for receiving the tube. The method may further include moving a bar of a clamp from a disengaged position to an engaged position to secure the tube within the groove, the bar including a pad on an inner surface configured to lie in a channel transverse to the groove in the engaged position. In some implementations, the method may include pivoting the bar about a hinge axis substantially parallel to the groove. In other implementations, the method may include securing the bar with a closure mechanism such as a snap-tab or bolt-and-eye arrangement. In some examples, the method may further include selectively adjusting the longitudinal position of a first tube received in a groove on a superior surface of the support while maintaining a second tube received in a groove on an inferior surface of the support in a secured position.

In some examples, repositioning the end portions within cheek pad channels by a distance X is accompanied by a corresponding repositioning of one or more tubes by the same distance X to maintain the desired placement. In some examples, when the end portions are advanced further into the cheek pad channels by a distance X, one or more tubes may be retracted by the distance X. In some examples, when the end portions are retracted within the cheek pad channels by a distance X, one or more tubes may be advanced by the distance X, thereby avoiding unintended removal or displacement. In some examples, one or more tubes may be adjusted before the end portions are repositioned. The distance X may correspond to the spacing between successive ribs on the end portions, which in some implementations is 0.25 cm, although other values of X are contemplated.

It should be understood that any of the example support apparatuses disclosed herein may be used in combination with any of the example cheek pad assemblies disclosed herein. For instance, while certain figures illustrate particular support apparatuses (e.g., 120, 220, 320, 420, 520, 620) in combination with particular cheek pad assemblies (e.g., 132, 232), the features of the apparatuses and cheek pads are not limited to those illustrated pairings.

It should be understood that the apparatuses and methods disclosed herein may be adapted for use with various patient populations, including neonates, pediatric patients, and adults, and may be scaled or configured accordingly. The disclosed features may also be implemented in single-use disposable devices or reusable devices.

Although the different examples are illustrated as having specific components, the examples of this disclosure are not limited to those particular combinations. It is possible to use some of the components or features from any of the embodiments in combination with features or components from any of the other embodiments.

The foregoing description shall be interpreted as illustrative and not in any limiting sense. A worker of ordinary skill in the art would understand that certain modifications could come within the scope of this disclosure.

What is claimed is:

1. A tube support apparatus, comprising:
a brace for attachment to a patient;
a support extending from the brace and including a surface providing a groove for receiving a tube and a channel transverse to the groove; and
a clamp comprising a bar for securing the tube within the groove, wherein the bar includes a pad on an inner surface of the bar positioned to lie in the channel when the bar is in an engaged position.

2. The tube support apparatus of claim 1, wherein the brace includes a curved support portion having opposed ends, each end received in a respective pad assembly adhered to the patient.

3. The tube support apparatus of claim 1, wherein the support includes superior and inferior surfaces, each surface providing a groove for receiving respective tubes.

4. The tube support apparatus of claim 1, wherein the bar is pivotable about a hinge axis substantially parallel to the groove.

5. The tube support apparatus of claim 1, wherein the clamp comprises a polymeric material.

6. The tube support apparatus of claim 1, wherein the support includes a superior surface providing a plurality of grooves for receiving respective tubes and an inferior surface providing a plurality of grooves for receiving respective tubes, such that a tube on one surface can be adjusted while a tube on the opposite surface remains secured.

7. The tube support apparatus of claim 6, wherein the grooves on the superior surface are sized differently from the grooves on the inferior surface to accommodate tubes of different diameters.

8. The tube support apparatus of claim 6, wherein the clamp includes the bar configured to secure a tube within the groove on the superior surface and a second bar configured to secure a tube within a second groove on the inferior surface, the bar being movable independently of the second bar.

9. The tube support apparatus of claim 8, wherein movement of the bar allows selective release or adjustment of a tube in a groove of the superior surface while a tube in the second groove of the inferior surface remains secured.

10. The tube support apparatus of claim 1, wherein the brace comprises a curved portion contoured to complement the patient's face.

11. The tube support apparatus of claim 1, wherein the clamp includes the bar configured to secure a tube within a groove on the superior surface and a second bar configured to secure a tube within a groove on the inferior surface, the bars being pivotable about hinge axes substantially parallel to the grooves and independently movable to selectively release or adjust a tube.

12. The tube support apparatus of claim 1, further comprising a second groove on the support and a second channel transverse to the second groove, and wherein the clamp further comprises a second bar for securing a tube within the second groove, the second bar including a second pad configured to lie in the second channel when the second bar is in an engaged position.

13. The tube support apparatus of claim 12, wherein the bar and the second bar are pivotable about hinge axes substantially parallel to the grooves and independently movable to selectively release or adjust a tube.

14. The tube support apparatus of claim 1, wherein the brace includes a curved support portion with opposed ends received in cheek pad assemblies adhered to the patient, the support further including a second groove on an opposed surface and a second channel transverse to the second groove, and the clamp further including a second bar for securing a tube within the second groove, the second bar including a second pad on an inner surface of the second bar positioned to lie in the second channel when the second bar is in an engaged position, the bar and the second bar being pivotable about hinge axes substantially parallel to the first and second grooves and independently movable to selectively release a tube secured by one of the first bar and the second bar while another tube secured by the other of the first bar and the second bar remains secured.

15. The tube support apparatus of claim 1, wherein the pad comprises an elongated precut self-adhesive piece of medical foam rubber.

16. The tube support apparatus of claim 1, wherein the pad comprises a compressible material selected from foam, gel, silicone, rubber, or elastomer, configured to conform to an outer surface of the tube.

17. The tube support apparatus of claim 6, wherein a first tube is secured in a groove on the superior surface and a second tube is secured in a groove on the inferior surface, the clamp being configured such that the first tube can be adjusted longitudinally while the second tube remains fixed.

18. The tube support apparatus of claim 1, wherein the support and the clamp are molded as a single piece with the bar formed as a living hinge.

19. The tube support apparatus of claim 1, wherein the bar is secured in the engaged position by a bolt and eye arrangement, the bolt being provided on one of the support and the bar and the eye being provided on the other of the support and the bar.

20. The tube support apparatus of claim 1, wherein the bar is secured in the engaged position by a bolt extending from the support and received in an eye formed in the bar.
